(19) United States
(12) Patent Application Publication
(10) Pub. No.: US 2009/0036541 A1
(43) Pub. Date: Feb. 5, 2009

(54) Applicator and chemical combination for better topical anesthesia

(76) Inventor: Donald David Mardis, Denver, CO (US)

Correspondence Address: Donald David Mardis, 930 Holly St., Denver, CO, 80220, US

(21) Appl. No.: 11/897,682

(22) Filed: Aug. 30, 2007

Related U.S. Application Data
(60) Provisional application No. 60/963,136, filed on Aug. 2, 2007.

(51) Int. Cl. A61K 31/10; A61M 35/00; A61P 23/00

(52) U.S. Cl. 514/708; 604/289; 604/290

(57) ABSTRACT

One embodiment of a thin plastic applicator (1) containing a thinner plastic bubble (2) adhering to the inner surface (3) and containing a solution of dimethyl sulfoxide with one or more local anesthetic agent. A thick plastic rib (4) provides reinforcement, separating two textured sides (5) for ease of handling. A thick plastic rim (6) is attached to an adhesive foam ring and covered with protective paper (7). Removing this paper, the applicator is adhered to a disinfected and dry treatment site, and squeezed to break the inside bubble. After sufficient time to allow penetration of the anesthetizing agent, the bubble is removed, and medication or additional anesthetic injected painlessly. This replaces currently employed less effective methods of topical anesthesia, and provides health care professionals a means of fear and pain reduction where before none was available.

### CROSS REFERENCE TO RELATED APPLICATIONS

This application claims the benefit of provisional patent application Ser. No. 60/963,136 filed Aug. 2, 2007 by the present inventor.

### FEDERALLY SPONSORED RESEARCH

Not applicable

### SEQUENCE LISTING OR PROGRAM

Not Applicable

### BACKGROUND

1. Field of Invention

This invention relates to topical anesthesia, specifically to better anesthetizing chemical combinations and an applicator for said combinations.

2. Prior Art

Health care professionals around the world perform invasive procedures every day. Such procedures produce pain of varying severity, requiring needle-injection of anesthetic agents beforehand. Approximately half of the population temporarily or permanently avoids said procedures due to the fear of pain of the injection. This adversely affects the health and productivity of society.

Patients of any age, who do receive care, experience anxiety and fear based on past injections and the frightening anecdotes of other people. Upon feeling the needle, patients may move involuntarily and suddenly, often causing injury to themselves or the professional. If delivery of the anesthetic is thus altered, it would require additional injections, repeating the cycle of events. This affects negatively the relationship which health care professionals have with the very patients they are committed to healing.

Presently, the only topical anesthetic used is benzocaine, as a gel or an atomized spray. Because of the mostly-impermeable nature of skin, it is used only in dentistry where it is slightly effective (although extremely superficially) on exposed mucous membranes. Dentists admit that it is mainly a “psychological” benefit and their patients will admit that it “is better than nothing”. For dentists, the lack of a true topical anesthetic remains the linchpin in widespread treatment of the most common bacterial infection in the human population: dental caries (aka. decay or cavities).

Dimethyl sulfoxide (DMSO), a wood-industry by-product used to dissolve non-water-soluble solids, has been in commercial use for over half a century. To this day, high school and college chemistry students utilize it to introduce chemicals into liquid solution which cannot be studied otherwise.

Many therapeutic uses, both homeopathic and USFDA-approved, have been identified for DMSO. In 1961, Dr. Stanley Jacob, head of the organ transplant program at Oregon Health Sciences University, discovered DMSO's skin-penetrating properties and began a lifelong investigation and promotion of it's potential health benefits.

Seven years later, reported in the Annals of the New York Academy of Sciences, Brechner, Cohen, and Pretsky reported on skin anesthesia with tetracaine dissolved in DMSO. This anesthetic is ten-fold as potent as lidocaine, yet has a much-delayed onset time to take effect. On skin, loss of feeling came in as much as 45 minutes, but lasted for several hours. Some practicing plastic surgeons rely on a mixture of lidocaine and prilocaine to anesthetize areas for skin grafts, debridement of ulcers, and chemical peels. Said doctors wait for over 2 hours for this preparation, known as EMLA cream, to take effect, although many patients alternatively “tough it out” in the search for personal cosmetic perfection.

In 1989, The United States Patent and Trademark Office granted U.S. Pat. No. 4,851,442 to Watson, for a preparation of DMSO with water, lidocaine hydrochloride, and citric acid for the treatment of arthritic calcium deposits, inflammation and pain. Unfortunately, as the acid-base of the human body is nearly neutral, the mistakes (repeated to the current day) of making anesthetics soluble-in-water as their hydrochloride salt were duplicated. Despite the low-cost advantage of combining chemicals with simple water in medicines, current local anesthetics produce a feeling of “burning” and “pain” when injected at any rate under skin or mucosa, due to the human body's reaction to non-neutral chemical combinations.

Then in 2004, the USPTO awarded U.S. Pat. No. 6,790,855 to Pasternak & Kolesnikov and their novel approach to peripheral pain management. These researchers sought to alleviate the intractable pain endured by cancer patients through the combination of morphine and lidocaine in varying percentages. As with earlier developments, they soaked a gauze with the solution and taped said gauze onto the target site for days or weeks as the treatment.

### SUMMARY

In accordance with one embodiment, an applicator is roughly hemi-spherical in shape with reinforcing rims at the “cut” surface and the equatorial rib. While the adhesive ring is covered with a protective paper covering, inside is an easily-burst plastic bubble adhering to the inner applicator surface, containing the simple combination of local anesthetic, dimethyl sulfoxide, and water and vasoconstrictor (with preservative) as needed.

### DRAWINGS—FIGURES

FIG. 1A is a side view and FIG. 1B is a “cut”-surface view of a first embodiment of my invention.

### DRAWINGS—REFERENCE NUMERALS

| | | | |
|---|---|---|---|
| 1 | hemi-sphere | 2 | inner bubble |
| 3 | inner surface | 4 | reinforcing rib |
| 5 | textured outer surface | 6 | adhesive on reinforced rim |
| 7 | protective paper cover (removed) | | |

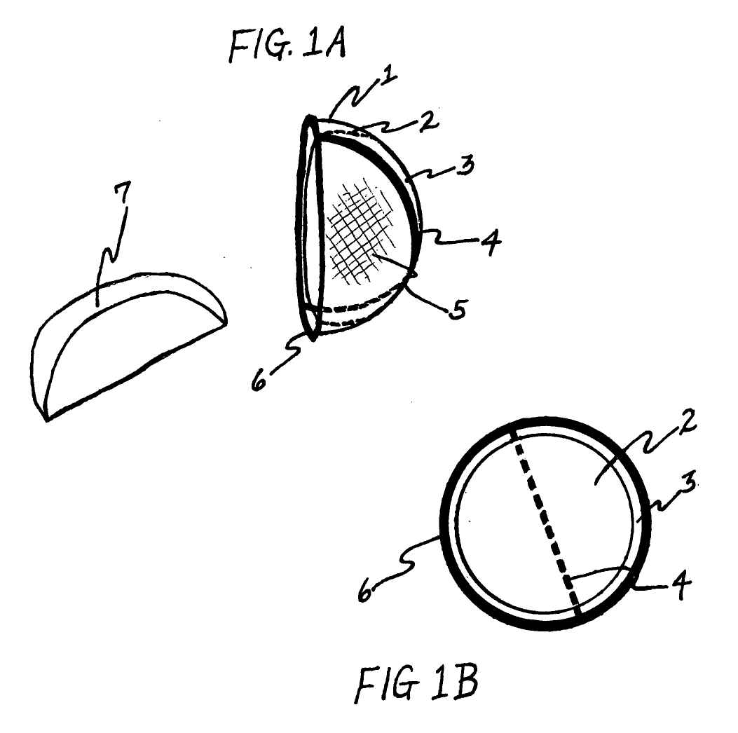

### DETAILED DESCRIPTION—FIGS. 1A AND 1B—PREFERRED EMBODIMENT

One embodiment is illustrated in FIG. 1A (side view) and FIG. 1B (“cut”-surface view). The hemi-sphere 1 consists of a thin plastic with a thinner-still inner plastic bubble 2 adhering to it's inner surface 3. A rib 4 of thick plastic crosses at any diameter of the hemi-sphere, separating on each side a textured outer surface 5 for easier handling with fingers or a hemostat instrument. The rim 6 is also of thick plastic and is coated first with a double-sided high-tack transfer adhesive and then an adhesive foam tape, both available from 3M Health Care Medical Specialties products. The adhesive is protected before use by a paper cover 7. The hemi-sphere itself is made by FormTight, Inc., in Denver, Colo. The anesthetic and dimethyl sulfoxide is available through Spectrum Pharmacy Products, Tucson, Ariz. The inner bubble began using a “bubble wrap” product from the Sealed Air Corporation, and contains approximately 0.22 milliliters of 100% dimethyl sulfoxide with 16% lidocaine (equal to 16 milligrams per 100 milliliters of DMSO). This bubble contains the same amount of one cartridge of standard lidocaine anesthetic which a dentist would inject with a needle.

### Operation—FIGS. 1A and 1B

A health care practitioner begins by selection of a site to be anesthetized. After applying a surface disinfectant, the area is dried. One applicator is held gently via the textured surface, the paper removed, and pressed onto the site. After several seconds and a slight tug-back to test for adhesion, the applicator is compressed just enough to burst the inner bubble. The applicator is released and left for the required time. On mucous membrane, only several minutes is required for onset, but four to 5 times that may be required for skin anesthesia. After this, the applicator is removed and additional local anesthetic may be injected without causing pain.

### Advantages

Medical use of a topical anesthetic on skin is virtually unheard-of. Unfortunately, for both adults and children, needles must be used in vaccinations, starting intravenous administration of medications and fluids, blood draws for sampling or donation, and home administration of hormones like insulin and testosterone. These and “minor” procedures like suture removal and ingrown-toenail cleansing could be made to feel more comfortable with less apprehension or fear. Now, they are just asked to “tough it out”.

Current dental use of benzocaine is limited in two respects. First, the “technique” amounts-to placing a Q-tip with a 20% gel form of the anesthetic, which then is diluted & made ineffective by saliva and moving by the tongue or cheeks. Second, the anesthetic only barely penetrates the surface, and is broken down by enzymes in a short period due to the limited amount present. Both the male and female external sex organs AND the area surrounding the external anus (including hemorrhoid-prone blood vessels) are also mucous membranes subject to pre-injection numbing with my invention.

My invention is an improvement in these ways:

(a) The most advantageous benefit is both true and psychological. When a patient is undergoing pre-injection anxiety, they will feel the numbing effect. When there is no pain later from the needle, they will understand the health care professional is doing everything possible to truly care for them.

(b) The shape and physical presence will provide a calming effect, perceived as a new development in reducing pain.

(c) Cost will be comparable to current local anesthetic, plus the cost-savings of the loss of benzocaine. As news spreads, those who avoided past treatment will seek care, increasing market size for health care professionals.

### Description—Alternative Embodiments

While the prototype is mostly clear and approximately 1 centimeter in diameter, other sizes, colors, and shapes could be developed based on intended purpose. Similarly, the percentage of dissolved local anesthetic and volume of DMSO may be varied. For applications on facial skin, water or other agents may be added to reduce drying and redness after treatment. If enough water is used, the water-soluble salt of the anesthetic may be added to maintain effectiveness. Vasoconstrictors (with preservative) could be added to extend duration of numbness and to provide ease of site identification.

### Conclusions, Ramifications, and Scope

Accordingly, the reader will see that, according to one embodiment, my invention provides an applicator and a better topical anesthetic for general use in medicine, dentistry, and veterinary procedures causing pain and fear. Patients could use it at home, just as they are trained to administer insulin and other medications, perhaps making them more compliant and reducing medical costs through uncontrolled disease.

While my description contains specificities, these should not be construed as limitations on the scope of the invention, but rather as an exemplification of one preferred embodiment thereof. Many other variations are possible in color, size, shape, and chemical combination, especially with respect to the type of local anesthetic, depending on the intended use. For example, lidocaine may be substituted or combined with mepivacaine, etidocaine, bupivicaine, prilocaine, benzocaine, tetracaine, or any other derivations of cocaine, or they with each other. Also, as in rare cases of absolute allergy to these, an anti-histamine may be substituted or added to cause topical anesthesia.

Thus the scope of the embodiment should be determined by the appended claims and their legal equivalents, rather than by the examples given.

I claim:

1. An applicator for containment, transport and confinement during administration of a superior topically acting anesthetic solution.

2. A method of providing topical anesthesia to a subject comprising topically administering to peripheral sites in the subject a pharmaceutical composition of dimethyl sulfoxide solvent with varying dilutions of water and necessary agents to address local side effects and with varying concentrations of local anesthetic alone or in combination with each other.
